SEQUENCE LISTING (4 sequences)

SEQ ID NO: 1 - DNA, 10 bp, Artificial Sequence
  Description of Artificial Sequence Synthetic oligonucleotide
gaannnnttc

SEQ ID NO: 2 - DNA, 9 bp, Artificial Sequence
  Description of Artificial Sequence Synthetic oligonucleotide
nncastgnn

SEQ ID NO: 3 - DNA, 17 bp, Artificial Sequence
  Description of Artificial Sequence Synthetic oligonucleotide
cctacgggaggcagcag

SEQ ID NO: 4 - DNA, 19 bp, Artificial Sequence
  Description of Artificial Sequence Synthetic oligonucleotide
ggactaccagggtatctaa

(19) United States
(12) Patent Application Publication
(10) Pub. No.: US 2015/0152483 A1
(43) Pub. Date: Jun. 4, 2015

(54) FAST AND RELIABLE WINE LACTIC BACTERIA IDENTIFICATION METHOD

(71) Applicant: UNIVERSIDAD DE CHILE, Santiago Santiago (CL)

(72) Inventors: Jaime Moises Romero Ormazabal, Santiago (CL); Carolina Alejandra Ilabaca Diaz, San Miguel (CL)

(21) Appl. No.: 14/395,276

(22) PCT Filed: Apr. 8, 2013
(86) PCT No.: PCT/IB2013/052802

§ 371 Date: Oct. 17, 2014

(30) Foreign Application Priority Data
Apr. 19, 2012 (CL) 2012-00993

(51) Int. Cl. C12Q 1/68

(52) U.S. Cl. CPC C12Q 1/689; C12Q 2600/158

(57) ABSTRACT

The invention depicts a method to identify the Oenococcus, Leuconostoc, Pediococcus and Lactobacillus bacteria in a fermentation sample which comprises the steps of a) obtaining a sample from a fermentation; b) eliminating the colorants, phenols and other substances that inhibit the PCR of the sample; c) lysis of the bacteria present in the sample; d) DNA extraction from the samples; e) PCR amplification of the DNA extracted using universal primers designed for a target region of the 16S rRNA gene, f) digestion of the amplified using a restriction solution with enzymes selected by theoretical analysis; g) incubation and h) electrophoresis of the fragments obtained. The invention also refers to a kit for implementing the method described.

### FIELD OF THE INVENTION

This invention relates to the enology industry, especially to the microbiological control and monitoring of the bacterial microorganisms during processes such as the alcoholic fermentation and the malolactic fermentation for different varieties of wines (red, white rose and sparkling) the elaboration of fruit ferments (grapes and others) for the production of cider, chicha and pajarete among others, the elaboration of vinegars and the elaboration of ferments to produce wines and/or base ferments for distillates such as pisco, rum, whisky, brandy, grappa, among others because it allows identifying the presence of the four more common bacteria that participate in some of the fermentations in these processes.

### BACKGROUND OF THE INVENTION

Lactic bacteria are very important microorganisms in enology that may favor the quality of wine and other fermented products. Anyhow, some of these bacteria can generate unwanted and even toxic metabolites such as the biogenic amines like the histamine. These lactic bacteria are fundamental in the malolactic fermentation, anyhow the monitoring and microbiological controls necessary in this stage of the production have been delayed because the culture of these bacteria in conventional media is difficult. Lactic bacteria may be present in a large number of fermentation processes, for example in the malolactic fermentation. Malolactic fermentation is part of red wines production process, anyhow it is done without a control protocol or microbiological diagnosis that may allow taking the corrective actions and integrate the handling and microbiologic diagnosis to the process, in order to take advantage of the microorganisms and reduce the harmful effects.

In enology it is of great importance to avoid microbiological issues during fermentations. In wines and other liquors production, the alcoholic fermentation is done with yeasts while the malolactic fermentation happens through the action of the lactic bacteria or lactic acid bacteria (BL). The main effect of the malolactic fermentation in the production of wines and other liquors is the reduction of the acidity (generally with a pH below 3.5) by means of transforming the malic acid in lactic acid. In liquors with high acidity, the malolactic fermentation is desirable to improve the product. Moreover the BL may influence the organoleptic properties of the wine, particularly the aromatic properties by affecting the contents of polyphenols and other important components. It is of vital importance to know and control on time the bacterial population present in the malolactic fermentation because unexpected changes in the microorganisms or an imbalance of microbial populations may produce important alterations in the final products (wines and other liquors).

There are many techniques that allow identifying lactic bacteria present in the liquor production processes; the problem with these techniques is that most of them need a culture stage which at least takes five to eight days so the identification of the bacteria acting in the fermentations is delayed. At this point it is important to say that the different genders of lactic bacteria require different culture media and incubation conditions (microaerophilic) for its optimal growth. For example, for Oenococcus a very specific and rich medium is needed such as the MLO medium. In general in order that the lactic bacteria of different gender grow a base medium is needed that must be supplemented with different amino acids and/or sugars. This culture requires days of incubation (more than four days) to have bacterial growth (colonies). This step of seeding in culture medium prevents from having an almost instantaneous vision of the bacteria that are acting in the fermentation and take corrective action. Moreover, it is essential considering that the path through a culture medium limits the identification of the population present in a fermentation; different studies have shown that part of the bacterial population present in wine fermentations that is viable in the medium (process) but not possible to be cultured in specific media. The consequence is that the population obtained in culture media may represent at least 1% of the total bacteria present in a fermentation sample. This means that many bacteria that were originally present may be excluded from the analysis whereas they might potentially represent more than 90% of the bacterial populations in one sample. This is explained because many bacteria can be in a viable form and acting in one environment although at the moment of them being seeded in some culture medium they are not able to grow and form colonies (concept of viable but not possible to be cultured).

The present invention proposes a molecular analysis that is way superior to the existing one and is simple to implement, therefore it may be used in a wide range of service laboratories in the enology industry around the world.

### STATE OF THE ART

There are several papers characterizing and identifying lactic bacteria (BL), not only those present in enology fermentations but also in different environments like milk, yoghourt, pickles, etc. A large number of scientific publications consider the identification of these bacteria, most of them are based on the analysis of a specific region of the DNA, either for different genders (16S-ARDRA, a Tool for Identification of Lactic Acid Bacteria Isolated from Grape Must and Wine; Rodas, A. M.; Ferrer, S.; Pardo, I.; Syst Appl Microbiol 2003 September; 26(3):412-22) and for a specific species (Typification of Oenococcus oeni strains by multiplex RAPD-PCR and study of population dynamics during malolactic fermentation; Reguant, C.; Bordons, A.; J Appl Microbiol. 2003; 95(2): 344-53).

Rodas et al. (16S-ARDRA, a Tool for Identification of Lactic Acid Bacteria Isolated from Grape Must and Wine; Rodas, A. M.; Ferrer, S.; Pardo, I.; Syst Appl Microbiol. 2003 September; 26(3):412-22) describes the identification of musts and wines bacteria through PCR amplification (polymerase chain reaction) of the 16S rDNA and the digestion with enzymes. What is particular in this description is that in order to run this amplification colonies obtained through culture with specific media are used.

Reguant and Bordons (Typification of Oenococcus oeni strains by multiplex RAPD-PCR and study of population dynamics during malolactic fermentation; Reguant, C.; Bordons, A.; J Appl Microbiol. 2003; 95(2); 344-53) describes a PCR method to amplify at random based in supposed polymorphisms (RAPD-PCR randomly amplified polymorphic DNA), that generates unique and discriminating DNA profiles to identify Oenococcus oeni.

Namelli el al. (Determination of lactic acid bacteria producing biogenic amines in wine by quantitative PCR methods; Namelli, F.; Claisse, O.; Gindreau, E.; de Revel, G.; Lonvaud-Funel, A.; Lucas, P. M.; Lett Appl Microbiol. 2008 December; 47(6); 594-9) describes methods of fast numbering for lactic bacteria producing biogenic amines in wines. The methods are based on conventional and quantitative PCR to detect genes producing biogen amines such as histamine and putrescine.

Ruiz el al. (Intraspecific genetic diversity of lactic acid bacteria from malolactic fermentation of Cencibel wines as derived from combined analysis of RAPD-PCR and PFGE patterns; Ruiz, P.; Izquierdo, P. M.; Seseña, S.; Palop, M. L.; Food Microbiol. 2008 October; 25(7): 942-8) uses three techniques to analyze the diversity of the lactic microbiota responsible for the spontaneous malolactic fermentation in Cencibel wines. The techniques are the randomly amplified polymorphic DNA PCR (RAPD-PCR), pulsed field gel electrophoresis (PFGE) and differential display polymerase chain reaction (DD-PCR). The authors recognize deficiencies in the possibility of reproducing the DD-PCR and that for a better resolution the PFGE and RAPD data should be combined.

Claisse et al. (Differentiation of wine lactic acid bacteria species based on RFLP analysis of a partial sequence of rpoB gene; Claisse, 0; Renouf, V.; Lonvaud-Funel, A.; J. Microbiol Methods. 2007 May; 69(2); 387-90) run a polymerase chain reaction-restriction fragment length polymorphism (PCR-RFLP) analysis of rpoB sequences to identify lactic bacteria species commonly isolated from wine.

Neeley et al. (Differential real-time PCR assay for enumeration of lactic acid bacteria in wine; Neeley, E. T.; Phister, T. G.; Mills, D. A.; Appl Environ Microbiol. 2005 December; 71(12): 8954-7) describes an assay of two PCRs in real time to list the total population and the lactic “non-oenococcal” bacteria population in musts and wines in order to assess the deterioration danger caused by these bacteria.

Du Plessis et al. (Identification of lactic acid bacteria isolated from South African brandy base wines; du Plessis, H. W.; Dicks, L. M.; Pretorius, I. S.; Lambrechts, M. G.; du Toit, M.; Int J Food Microbiol. 2004 Feb. 15; 91(1): 19-29) describes the use of sequence analysis of 16S rRNA and PCR using species-specific primers to identify lactic bacteria strains present in fruit musts in different status of production of wines used as brandy base in South Africa.

Cho et al. (Development of a quantitative PCR for detection of Lactobacillus plantarum primers during wine malolactic fermentation; Cho, G. S.; Krauss, S.; Iluch, M.; Du Toit, M.; Franz, C. M. A. P.; Journal of Micriobiology And Biotechnology 21 (12): 1280-1286 December 2011) diclose a PCR quantitative method (qPCR) to detect Lactobacillus plantarum during the malolactic fermentation of Grauburgunder wine. The PCR is specific to one strain (IWBT B 188). The technique described in this paper is specific and unique for this strain, therefore a general view to the microbial composition existing in a sample cannot be done.

Cremonesi et al., (Development of a pentaplex PCR assay for the simultaneous detection of Streptococcus thermophilus, Lactobacillus delbrueckii subsp bulgaricus, I. Delbrueckii subsp. Lactis, I. Helveticus, I. Fermentum in whey primer for grana padano cheese; Cremonesi, P.; Vanoni, L.; Morandi, S.; Silvetti, T.; Castiglioni, B.; Brasca, M.; International Journal of Food Microbiology; 146 (2): 207-211 Mar. 30, 2011) describes the development of a detection method for five bacteria simultaneously in whey primer for grana padano cheese. Target sequences were a group of genes coding for the production of beta-galactosidase (for S. thermophilus and L. Delbrueckii sp bulgaricus); for the proteinase synthesis associated to the cell wrapping (for L helveticus); for the production of the dipeptide transport system (for L. delbrueckii sp lactis) and for arginine ornithine transport protein (for L fermentum). An enrichment of the whey samples is done for this method before the extraction of the nucleic acids. This enrichment is an overnight incubation that delays the detection in at least 12 hours. The technique is also difficult to implement because each bacterium needs a couple of specific primers.

There are also patent applications related to identify methods for lactic bacteria. WO2008003811 relates to a method to identify and simultaneously and specifically detect lactic bacteria and bifidobacteria in fermented milk and primer cultures of fermented milks. The method uses specific primers for the 16S rRNA gene of lactic bacteria and for the transaldolase gene of bifidobacteria. The method is complemented using denaturing gradient gel electrophoresis of polyacrylamide (DGGE) to separate the amplicons.

KR100774540 describes a method to identify lactic bacteria at gender level in kimchi (fermented food, typical Korean food) and a method to determine the size of the population in order to produce kimchi with uniform quality. Specific primers are used for Lactobacillus sp., Weissella sp., Pediococcus sp., and Leuconostoc sp.

KR100775641 describes a method to identify lactic bacteria in probiotic products using multiple PCR in order to distinguish among two types of Lactobacillus sp and two types of Bifidobacterium sp. through a reaction, thereby improving the fastness and precision of the identification. Eight different primers are used to identify the four bacteria.

CN102226142A, discloses the use of PCR-DGGE to identify the functional microorganisms that take part in the fermentation of rice vinegar. They also obtain isolated cultures of the microorganisms and they can strengthen the fermentation process by adding defined inocula. The method allows the shortening of fermentation time, increasing the control and quality of the product and improving the use of the raw material.

CN102242193A discloses the use of DGGE to classify the microorganisms in fermented milk, wine and animal micro ecologic preparations.

CA2385652C relates to a method to detect important microorganisms for the production of beer. The invention focuses in the development of a fast method to detect the contamination with microorganisms in beer or raw material for the production of beer. The method consists of amplifying a sample through PCR and then hybridizing with all the important microorganisms with a specific fragment in the production process of beer. The final step consists of the detection of hybrid nucleic acid.

Some of the former documents mention the DGGE technique to identify the microorganisms participating in fermentations. This technique allows separating or differentiating the microorganisms present in a sample, although it has a significant difficulty in the preparation of the analysis (large size gels and complex composition) and the analysis of results which although is not complex, tools such as specific programs for the analysis of banding patterns, handling of web databases, etc. should be used. The DGGE technique is not an easy technique to implement neither is it friendly for a day-to-day use. Among others, it requires an equipment of high relative cost and highly qualified personnel to do it. It also has the disadvantage that it only separates the gender or bacterial species based on the % GC and therefore it does not show the presence of a specific sequence (gender or species). Consequently a band may contain more than one bacterium which makes the application difficult.

Other documents mention the RAPD-PCR technique that consists of amplifications on “unspecific” regions in the genome of these bacteria. Therefore this technique is sensitive to the quality of the DNA obtained, its fragmentation, purity, etc. because this may affect the efficiency of the amplification and thereby show false differences among the isolated ones.

The documents previously described are those considered to be the closer ones to the state of the art. The studied scientific and patents literature show that recent efforts in this subject have focused on obtaining methods that allow the fast and efficient detection of various species of Lactobacillus, Oenococcus and other bacteria present in wine and other fermented products. The PCR in this field is widely used for these purposes.

The closest documents to the state of the art do not specifically describe the detection of Oenococcus, Leuconostoc, Pediococcus and Lactobacillus they neither use exactly the same methodology with conventional PCR.

There are many differences among these papers, other similar ones and this invention: these papers use methodologies based on an identification procedure that requires pure strains or isolated in specific culture media, which greatly delays the procedure. The method of this invention is based on the analysis of bacterial nucleic acids so it is independent of the capacity of recovering the bacteria in a culture. This represents several advantages: a significant analysis time saving; a larger coverage of genders and species analyzed; a reduction in mistakes derived from the scarce culture capacity of the BL present (the culture can only recover 1% of the total). The method of this invention is independent from the culture therefore it saves time and thereby it is not subject to the mistake of examining just one small percentage of bacteria represented of the sample (1%). On the other hand, as a consequence of the isolation and work with pure strains, the other methods propose methodologies that are good to compare patterns in purity conditions or just useful when there is one single bacteria. The method of this invention is capable of identifying the bacteria in complex mixtures so it is not necessary to separate them either through culture or another method. Moreover, other methods require specific primers and they detect just one of the bacteria of interest. The technique of this invention is based on a target region (16S) which is amplified and then is differentiated via a restriction analysis without the need of a high cost sophisticated equipment. This invention is based on universal primers that consequently amplify the DNA of all the bacteria in the sample and uses the differences of the amplified sequences to reveal the presence of the bacteria of interest. As a consequence, the advantage is that it only requires one amplification reaction (PCR) per sample with a couple of universal primers because it can detect the most important genders (Pediococcus, Lactobacillus, Oenococcus and Leuconostoc) during the fermentation with lactic bacteria. This detection at a gender level is absent in the former document which does not include some of these genders that are very important, especially in the malolactic fermentation of wine.

Other methods may require complex detection or differentiation systems. The method of this invention just requires a conventional thermocycler for PCR and a common electrophoresis equipment. Even more, the method of this invention may quite fast deliver the microbiological diagnosis present in a sample which makes the methodology more friendly and easy to implement both in a winery and reference laboratories. The method of this invention also differentiates because it does not require an initial count because DNA is extracted directly from the fermentation sample without the need of knowing the amount of cells present in the sample. The technique of this invention is based on a target region (16S) that is amplified and then differentiated by a restriction analysis.

At the moment of presenting the identification techniques, mainly in the wine industry, these should be simple both to operate and to implement in the winery.

### BRIEF DESCRIPTION OF THE INVENTION

The invention describes a method to identify the main four genders of lactic bacteria that generally participate in the fermentations in the enology industry, especially in the malolactic fermentation of wine: Oenococcus, Leuconostoc, Pediococcus and Lactobacillus. The method consists of the extraction of DNA from a fermentation sample and the subsequent amplification with PCR with universal primers designed for a specific region of the gene 16S rRNA that amplify all the bacteria present in the sample. The amplified obtained are subject to digestion with restriction enzymes which generates the specific patterns for each bacterial gender allowing its identification. The method has the advantage of being fast and reliable and therefore it allows modifying the bacterial contents of the malolactic fermentation if necessary. The invention also provides a kit to apply the method in vineyards laboratories, fermented products plants, wineries and in reference laboratories of the enology and beer industry.

### BRIEF DESCRIPTION OF THE FIGURES

FIG. 1: Restriction enzymes used in the invention. Panel A is an E1 scheme that corresponds to a restriction enzyme recognizing the sequence GAANNNNTTC (ID SEC N° 1) and cleaves between the fifth and sixth nucleotide of the recognition sequence in both strands. Panel B is an E2 scheme that corresponds to a restriction enzyme recognizing the sequence NNCASTGNN (ID SEC N° 2) and cleaves the strand 5′-3′ two nucleotides after the underlined recognition sequence is finished and strand 3′-5′ two nucleotides before the underlined recognition sequence. N may correspond to A or C or G or T. S may correspond to C or G.

FIG. 2: Electrophoresis gel presenting the 16S rRNA region amplified using the pair of primers 341F (ID SEC N° 3) and 788R (ID SEC N° 4). The columns correspond to L: molecular weight marker BenchTop 100 bp DNA ladder, Promega; Lm: Leuconostoc mesenteriodes culture; Oo: Oenococcus oeni culture; Lb Lactobacillus brevis culture; Pp Pediococcus parvulus culture.

FIG. 3: Electrophoresis gel presenting the enzymatic digestion with the restriction enzymes selected theoretically applied in amplifications of standard strains cultures. The columns correspond to L: molecular weight marker GeneRuler™, Low range DNA ladder; Lm: Leuconostoc mesenteriodes culture; Oo: Oenococcus oeni culture; Lb: Lactobacillus brevis culture; Pp: Pediococcus parvulus culture.

FIG. 4: Electrophoresis gel presenting the enzymatic digestion with the selected restriction enzymes applied to samples of different stages in a wine fermentation. The columns correspond to L: molecular weight marker GeneRuler™, Low range DNA ladder; Lm: Leuconostoc mesenteriodes culture; Oo: Oenococcus oeni culture; Lb: Lactobacillus brevis culture; Pp: Pediococcus parvulus culture: 16; start of the alcoholic fermentation; 28 end of the alcoholic fermentation; 30 start of the malolactic fermentation; 6: half of the malolactic fermentation; 10: end of the malolactic fermentation.

### DETAILED DESCRIPTION OF THE INVENTION

The invention describes a method to identify the four main lactic bacteria genders that generally participate in the enology fermentations, especially in the malolactic fermentation of wine: Oenococcus, Leuconostoc, Pediococcus and Lactobacillus. The method is based on the analysis of the bacterial nucleic acids extracted and amplified directly from the fermentation samples independent of the growth in culture media. This provides a good advantage because it not only avoids time consumption typical of the microbial growth but it also is independent from the capacity of the microorganisms of growing in a culture medium. The former is very important because an important part of environment microorganisms, especially in the wine environment are not recovered in conventional culture media. Only small proportions of bacteria 0.1-10% are capable of growing in culture media and those who do it do not necessarily represent the most dominant genders and species in the sample, but they correspond to those that grow better in the conditions and culture medium used.

The identification method for lactic bacteria in fermentations, particularly malolactic, of this invention comprises the following steps: a) obtaining a sample from a fermentation; b) eliminating colorant substances, phenols and other inhibiting substances of the PCR of the sample; c) lysis of the bacteria present in the sample; d) DNA extraction from the samples; e) PCR amplification of the extracted DNA using universal primers designed for a target region of the 16S rRNA gene; f) digestion of the amplified using a restriction solution with enzymes selected by theoretical analysis; g) incubating; h) electrophoresis of the fragments obtained.

Step a: obtaining a sample from a fermentation may be done with any method known by one skilled in the art, provided that the sample is not contaminated during the process.

The sample may correspond to different fermentations, for example although not limited to: wine alcoholic fermentation (red, white, rose regular and sparkling), malolactic fermentation of wine (red, white, rose regular and sparkling), fermentation of fruit (grapes, apples and others) for the production of cider, chicha and pajarete among others, fermentation for the elaboration of vinegar (red wine, white wine, rice, apples, among others), fermentation of wines and base ferments of distilled liquors such as pisco, rum, whisky, brandy, grappa among others, fermentation of beer, among others.

In a preferred embodiment the sample corresponds to an alcoholic or malolactic fermentation of red, white, rose (regular or sparkling) wine. In an even more preferred embodiment, the sample corresponds to a red wine malolactic fermentation.

Step b) eliminating colorant substances, phenols and other inhibiting substances of the PCR of the sample is done with any method known in the art, for example, although not limited to: filtration, centrifugation, treatment with PVPP (polyvinyl polypyrrolidone), activated charcoal, bentonite, casein, silica, gelatin, agar, isinglass, albumen, gum arabic, vegetable proteins, silicon dioxide, chelant agents, enzymes or combinations thereof.

In a preferred embodiment step b) is done adding to the centrifuged sample of step a) a solution comprising PVPP (polyvinyl polypyrrolidone) at a concentration between 1 and 2% w/v in an adequate buffer. In a particular embodiment the adequate buffer is 0.1M EDTA, 0.15M NaCl.

Step c) lysis of the bacteria present in the sample is done for example, although not limited to, with the following methods: osmotic shock, mechanic homogenization (French press, blender, glass beads, embolus, among others) sonification, consecutive freezing and defrosting, treatment with detergents, with alkali, with enzymes or combinations thereof.

In a preferred embodiment step c) lysis of the bacteria present in the sample is done with a treatment with enzymes.

In a preferred embodiment step c) lysis of the bacteria present in the sample is done incubating the resulting sample of step b) with at least one glucohydrolase in an adequate buffer. In another preferred embodiment the glucohydrolase is lysozyme. In another preferred embodiment, step c) lysis of the bacteria present in the sample is done incubating the resulting sample of step b) with at least one protease in an adequate buffer. In another preferred embodiment, the protease is proteinase K. In an even more preferred embodiment, step c) lysis of the bacteria present in the sample is done incubating the resulting sample of step b) with at lease one glucohydrolase in an adequate buffer and then with at least one protease in an adequate buffer. In a preferred embodiment, the glucohydrolase corresponds to lysozyme and/or the protease corresponds to a proteinase K, independently one from the other. In an even more preferred embodiment, step c) lysis of the bacteria present in the sample is done incubating the resulting sample of step b) during at least 15 to 60 minutes at 35-37° C. with a solution that comprises a glucohydrolase, preferably lysozyme in an adequate buffer and then incubating the former during 15 to 60 minutes at 35-37 C with a solution that comprises a protease preferably proteinase K in an adequate buffer. In an even more preferred embodiment, the incubations are done during 30 minutes.

Step d) DNA extraction from the samples is done, for example although without limiting it to, with an extraction kit with the phenol chloroform method, with extraction methods and physical, chemical and/or enzymatic lysis using affinity resins by thermal shock with CTAB (Cetyl Trimethyl Ammonium Bromide)or combinations thereof.

In a preferred embodiment step d) DNA extraction from the samples is done preferably with an extraction kit.

In a preferred embodiment, step e) PCR amplification of the extracted DNA using universal primers designed for a target region of the 16S rRNA gene is done using primers 341F (5′-CCTACGGGAGGCAGCAG-3′) (ID SEC No. 3) and 788R (5′-GGACTACCAGGGTATCTAA-3′) (ID SEC No. 4) using regular conditions known by someone skilled in the art. In an even more preferred embodiment the conditions for the amplification are the ones described by Navarrete el al., 2010 (Navarrete P., Magne F., Mardones P., Riveros M., Opazo R., Suau A., Pochart P. and Romero J. 2010. Molecular analysis of intestinal microbiota of rainbow trout (Oncorhynchus mykiss) FEMS MICROBIOLOGY ECOLOGY Volume: 71 Issue: 1 Pages: 148-156).

In the amplification step the DNA of representative strains of the bacterial genders that recognize the method is also amplified. These DNA are also subject to the rest of the steps of the method in order to serve as comparison standards to identify each of the genders the method recognizes.

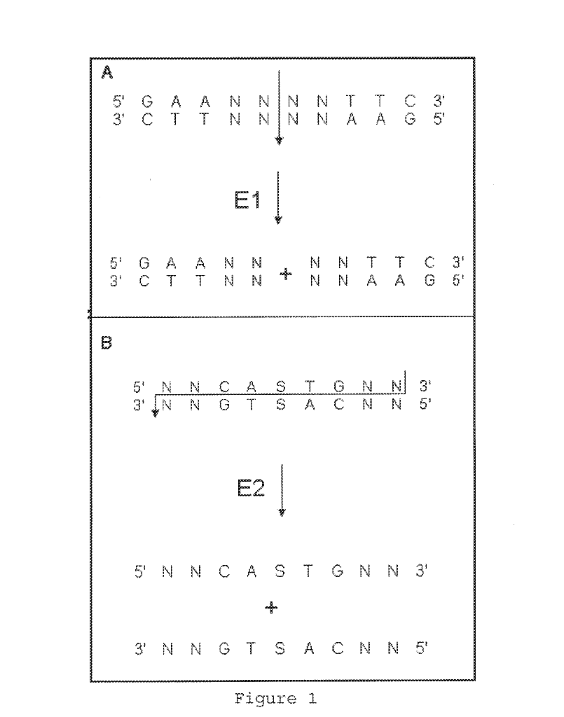

In a preferred embodiment, step f) digestion of the amplified using a restriction solution with enzymes selected by theoretical analysis is done with a solution that comprises differentiating enzymes that allow distinguishing unique profiles for Leuconostoc, Oenococcus, Lactobacillus and Pediococcus. The solution includes at least one enzyme corresponding to E1 and at least one enzyme corresponding to E2 as per FIG. 1. E1 is a restriction enzyme that recognizes the sequence GAANNNNTTC (ID SEC N° 1) and cleaves between the fifth and sixth nucleotide of the recognition sequence in both strands according to FIG. 1A. E2 enzyme is a restriction enzyme recognizing the sequence NNCASTGNN (ID SEC N° 2) and cleaves the strand 5′-3′ two nucleotides after the underlined recognition sequence is finished and strand 3′-5′ two nucleotides before the underlined recognition sequence according to FIG. 1B. The solution also comprises an adequate buffer where the enzymes are active.

Step g) incubating is done in order that the restriction enzymes may act. Therefore the conditions are such that the enzymes are active and cleave the amplified and can be defined by any expert in the field.

In a preferred embodiment step g) incubation, is done with a thermocycler with an appropriate program. In an even more preferred embodiment, the thermocycler program includes 90 to 120 minutes at 35-37° C. and 120 to 600 minutes at 62-65° C.

Step h) electrophoresis of the fragments obtained is done in order to visualize the fragments and compare them with the DNA standards of the representative strains of the bacterial genders that the method recognizes which are also subject to steps e) amplification, f) digestion and g) incubation.

In a preferred embodiment step h) electrophoresis of the fragments obtained is done in 8% to 10% polyacrylamide gels or gels of equivalent resolution. In another preferred embodiment the gels are subject to 70 to 100 volts during 60 to 90 minutes.

The method of this invention saves a large amount of time and allows obtaining a DNA sample that theoretically represents all microorganisms that are present in the sample, all at once, regardless of the ability to grow in conventional media. Therefore there is no need to seed in different selective culture media for different microorganisms. The technique developed is fast, easy and reliable for identifying Oenococcus, Pediococcus, Lactobacillus and Leuconostoc and for differentiating between these four bacteria that are normally found in enology fermentations, especially of wine.

This invention also provides a kit to apply the method in vineyards laboratories, breweries, wineries and of reference of the enology and brewery industry.

It should be considered that in a fermentation sample one, two or more lactic bacteria are possible to be found and that is where our invention is focused on, in a complex mixture.

These elements and the configuration of the process (protocol-methodology-controls-interpretation) are the base to establish a microbiologic diagnosis kit to identify the most common lactic bacteria in enology fermentations, especially in the malolactic fermentation of wine. This step is of great interest, especially in the handling of red wines and more recently for specific improvement in white wines such as the Chardonnay.

The kit of this invention comprises: i) solution or coloring medium; ii) At least one solution or lysis medium; iii) PCR master mix; iv) Restriction solution; v) DNA standards of strains of the bacterial genders that the method recognizes; vi) Instructions.

Additionally the kit of this invention may include media to obtain the sample from a fermentation.

The sample may correspond to different fermentations, for example although not limited to: wine (red, white, rose regular and sparkling) alcoholic fermentation; wine (red, white, rose, regular and sparkling) malolactic fermentation; fruits (grapes, apples and others) fermentation to produce cider, chicha and pajarete, among others, fermentation to produce vinegar (red wine, white wine, rice, apple, among others), wines fermentation and base ferments of liquors such as pisco, rum, whisky, brandy, grappa, among others, beer fermentation, among others.

The solution or the discoloring medium comprises at least a physical medium or a reactive that allows eliminating coloring substances, phenols and other PCR inhibiting substances. In a particular embodiment the solution or discoloring medium comprises at least one of the following physical media ∘ reactive: physical media to filtrate or centrifuge the sample, PVPP, activated charcoal, bentonite, casein, silica, gelatin, agar, isinglass, albumen, gum arabic, vegetable proteins, silicon dioxide, chelant agents, enzymes or combinations thereof.

In a preferred embodiment step b) is done adding to the centrifuged sample of step a) a solution comprising PVPP (polyvinyl polypyrrolidone) at a concentration between 1 and 2% w/v in an adequate buffer. In a particular embodiment the adequate buffer is 0.1M EDTA, 0.15M NaCl.

A solution or lysis medium adequate for the kit comprises at least one physical medium or a reactive that allows the cleavage of the cell wall and the bacteria membranes. In a particular embodiment the kit comprises at least a solution or lysis medium that comprises at least one of the following media or reactive: hypotonic solution, glass beads, detergents solution, hydrolytic enzymes solution or combinations thereof.

In an even more preferred embodiment, a solution or lysis medium of the kit comprises hydrolytic enzymes in an adequate buffer. In another even more preferred embodiment, a solution or lysis medium of the kit comprises at least one enzyme of the glucohydrolase type and/or an enzyme of the protease type with an adequate buffer. In an even more preferred embodiment, the glucohydrolase corresponds to lysozyme. In another preferred embodiment, the protease corresponds to proteinase-K. In a preferred embodiment, the kit comprises a glucohydrolase in a solution or lysis medium, preferably lysozyme in an adequate buffer and a protease preferably proteinase-K, en an adequate buffer. In another preferred embodiment the kit comprises at least two solutions or lysis medium, one comprising a glucohydrolase, preferably lysozyme in an adequate buffer and another one that comprises a protease, preferably proteinase-K, en an adequate buffer.

The PCR master mix of the kit comprises at least the universal primers 341F (ID SEC N° 3) and 788R (ID SEC N° 4). In a preferred embodiment the PCR master mix also comprises Mg, nucleotides and Taq polymerase enzyme in an adequate buffer.

The restriction solution of the kit comprises at least one enzyme corresponding to E1 and at least one enzyme corresponding to E2 as in FIG. 1 and an adequate buffer where the enzymes are active. E1 is a restriction enzyme that recognizes the sequence GAANNNNTTC (ID SEC N° 1) and cleaves between the fifth and sixth nucleotide of the recognizing sequence in both strands as per shown in FIG. 1A. Enzyme E2 uses a restriction enzyme that recognizes the sequence NNCASTGNN (ID SEC N° 2) and cleaves the strand 5′-3′ two nucleotides after the underlined recognition sequence is finished and strand 3′-5′ two nucleotides before the start of the underlined recognition sequence as shown in FIG. 1B.

The DNA standards of the kit of strains of the bacterial genders that the method recognizes comprise the DNA of at least one strain of each gender (Oenococcus, Lactobacillus, Leuconostoc and Pediococcus) and these strains correspond to species that are frequent in enology fermentations.

In a preferred embodiment the DNA standards of the kit of strains of bacterial genders that the method recognizes correspond to the strains Oenococcus oeni (JCM 6125), Pediococcus parvulus (NBRC 100673), Lactobacillus brevis (ATCC 14687) and Leuconostoc mesenteriodes (LMG 8159).

The instructions of the kit includes at least instructions referred to the incubations, digestion an amplification mix, PCR program, digestion conditions and/or electrophoresis.

The method and the kit of this invention have the advantages of being fast and reliable therefore they allow modifying the bacterial contents of the malolactic fermentation if necessary. The new technology (based on the direct examination of the DNA) allows through molecular methods, a fast identification (within hours) of the bacteria normally described in enology fermentations, especially malolactic fermentation that will be detected at the gender level avoiding the culture step or enriching the conventional media. The combination of restriction enzymes used for the digestion selected with an analysis of the fragments theoretically obtained provides the invention with the particularity that it is possible to identify the four genders in question in a single reaction thereby allowing corrections in the fermentation process. The lactic bacteria involved in these processes are difficult to culture and with the conventional methods they would be detected only after a week. During that time the corrections to the process are quite difficult because of the microbiological havoc of the process.

The method and the kit of this invention have clear advantages over what is known: I) rapid execution by avoiding the culture; II) easy execution since it does not require sophisticated equipment; III) certainty in the identification because it is based on band patterns that are compared with collection control strains which allow distinguishing and identifying the presence of any of the mentioned bacteria despite the mixtures because the resulting patterns are unmistakable, IV) it allows performing the analysis of the strains involved in the enology fermentations, especially in the malolactic fermentation with enough time to make corrections so as to have a better control of the process.

Advantages of the Process and of the Resultant Product

The method of this invention is independent from the culture therefore time is saved and thus it is not subject to the error of examining just a small percentage of bacteria represented of the sample (1%), it is capable of identifying the bacteria in complex mixtures, it is based on universal primers that in consequence amplify the DNA of all the bacteria in the sample; it uses the differences in the amplified sequences to reveal the presence of the bacteria of interest and only requires a conventional thermocycler for PCR and a regular electrophoresis equipment. Moreover, the method of this invention can deliver the microbiological diagnosis present in a sample in a relatively rapid way making the methodology more friendly and easy to implement both in vineyards, breweries and wineries and in reference laboratories.

### INDUSTRIAL APPLICATION

The method and the kit of this invention can be applied in the enology industry, specially in the control and monitoring of the alcoholic fermentation and the malolactic fermentation of wines of different varieties or presentations (red, white, rose, sparkling, etc.) in the production of fruit ferments (grapes and others) for the production of cider, chicha and pajarete among others, in the production of vinegars and in the production of ferments to produce wine and/or base ferments of distilled liquors such as pisco, rum, whisky, brandy, grappa, among others since it allows identifying the presence of the four most common bacteria that participate in some of the fermentations of these processes. The invention is also related to the brewery industry because it allows identifying the contamination by these bacteria during the production process. Particularly it may be applied in vineyards and wineries that want to have wines of good quality and in the laboratories of these companies or those who provide services to this industry as well as the reference laboratories for the analysis of the wine.

### EXAMPLES

### Example 1

### DNA Extraction from Wine Samples

The step of DNA extraction from wine samples that allows obtaining nucleic acids that can be amplified with PCR was expressed in the following steps: samples were taken and a treatment to eliminate coloring substances was applied before they were subject to the extraction. The treatment consisted of rinsing with PVPP (polyvinyl polypyrrolidone) 2% w/v (0.1M EDTA; 0.15M NaCl). Two rinses were done with this solution and then the protocol of the extraction kit PowerSoil was followed with previous incubation with lysozyme (20 mg/ml), followed by an incubation with proteinase K (20 mg/ml) both for 30 minutes at 37° C.

### Example 2

### Election of the Restriction Enzymes

The search of the combination of enzymes that differentiate the bacteria to be identified in a mixture is the result of an exhaustive and diligent work with bioinformatics elements. The step of the digestion with restriction enzyme that would allow distinguishing and identifying the four bacteria in question, was done with informatics analysis. The sequences known of the four bacterial genders in question were examined from the RDPii site, Ribosomal database Project II (with over a million 16S sequences) based on the sequences of the 16S rRNA ribosomal genes. Once these sequences were aligned a region to be amplified was defined based on sequences of consensus and in a theoretical way the bonding of the universal primers to be used in the assay was done. Once the fragment possible to be amplified was obtained a search of restriction enzyme was done (using the BioEdit program) that would be capable of providing different patterns among the four genders used. In this analysis available sequences of these four genders were used: 12 sequences were analyzed for Oenococcus, 86 sequences for Leuconostoc, 264 sequences for Lactobacillus and 55 sequences for Pediococcus. With this analysis a set of 2 enzymes was obtained that were capable of delivering different restriction patterns for the four genders. The enzymes chosen were E1 and E2, where E1 is a restriction enzyme that recognizes the sequence GAANNNNTTC (ID SEC N° 1) and cleaves between the fifth and sixth nucleotide of the recognition sequence in both strands as shown in FIG. 1A, and E2 is a restriction enzyme that recognizes the sequence NNCASTGNN (ID SEC N° 2) and cleaves the strand 5′-3′ two nucleotides after the underlined recognition sequence is finished and strand 3′-5′ two nucleotides before the underlined recognition sequence starts, as shown in FIG. 1B. The analysis of all the sequences analyzed is shown in Table 1.

TABLE 1
Summary of theoretical profiles using the selected
restriction enzymes El and E2 (FIG. 1) obtained with the
BioEdit program.

| | Pediococcus | | | |
|---|---|---|---|---|
| | Parvulus | Acidilactici | Damnosus | Pentosaceus |
| Profile | (9) | (18) | (12) | (16) |
| 51 65 79 127 144 | 100¹ | | 100 | |
| 51 65 66 78 79 127 | | 83.33 | | 93.75 |
| 51 65 78 79 193* | | 11.11 | | 6.25 |
| 51 66 78 79 192 | | 5.5 | | 6.25 |
| 51 78 79 258** | | 16.6 | | 6.25 |
| 66 78 79 116 127 | | 11.11 | | |
| 66 78 79 243 | | 5.5 | | |
| | Leuconostoc | | | |
| | Mesenteriodes | Pseudomesen | Citreum | Lactis |
| | (40) | (19) | (14) | (13) |
| 23 78 79 286 | 95 | 89.47 | 92.85 | |
| 23 78 365 | 2.5 | 5.26 | | |
| 78 79 309*** | 2.5 | 5.26 | 7.4 | |
| 78 101 286 | | | | 84.61 |
| 78 387 | | | | 15.38 |

| | Oenococcus |
|---|---|
| | Oeni (12) |
| 23 31 78 79 255 | 83.33 |
| 78 79 309*** | 16.66 |
| 31 79 101 255 | 8.33 |
| 79 123 255 | 16.33 |

| | Lactobacillus | | | | | |
|---|---|---|---|---|---|---|
| | Brevis | Delbrueckii | Fermentum | Casei | Plantarum | Hilgardii |
| | (29) | (25) | (78) | (27) | 104) | (1) |
| 51 65 78 79 | 95.55 | | | | | |
| 193* | | | | | | |
| 78 130 258 | 3.4 | | | | | |
| 51 78 79 258** | 3.4 | | | | 100 | |
| 65 78 79 130 | 3.4 | | | | | |
| 193 | | | | | | |
| 78 79 309*** | | 100 | 1.28 | | | |
| 51 79 336 | | | | | | 100 |
| 78 79 116 193 | | | 98.7 | | | |
| 58 78 137 193 | | | 1.28 | | | |
| 78 137 251 | | | 1.28 | | | |
| 78 79 135 174 | | | 1.28 | | | |
| 78 116 137 251 | | | 1.28 | | | |
| 51 65 79 271 | | | | | 0.96 | |
| 34 78 91 170 | | | | | 0.96 | |
| 193 | | | | | | |
| 34 70 91 271 | | | | | 0.96 | |

( )Number of sequences analyzed.
¹percentage of the profile within the sequences analyzed.
*Profile repeated in Pediococcus acidilactici and pentosaceus and Lactobcillus brevis and plantarum.
**Profile repeated in Pediococcus acidilactici and pentosaceus and Lactobacillus brevis and plantarum.
***Profile repeated in Oenococcus oeni; Lactobacillus delbrueckii and fermentum and Leuconostoc mesenteriodes, pseudomesenteriodes and citreum

### Example 3

### Confirmation of the Patterns Obtained In Silico Analyzing Reference Strains of the Lactic Bacteria Recognized by the Method

In order to contrast the patterns produced in silico, specifically 4 strains of known sequence of reference (International Microorganisms Collection, CETC) of the lactic bacteria that the method of the invention recognizes: Oenococcus oeni (JCM 6125) (the most important one in enology), Pediococcus parvulus (NBRC 100673), Lactobacillus brevis (ATCC 14687) and Leuconostoc mesenteriodes (LMG 8159).

The former strains were cultured in specific media MLO for Oenococcus oeni and MRS for the rest of the strains. The growing conditions for both culture media were at 28° C. for 48 hours in microaerophilic environment.

For the DNA extraction isolated colonies were re-suspended in PBS and incubated at 37° C. with lysozyme (20 mg/L) during 15 minutes and then with proteinase K (20 mg/L) during 60 minutes also at 37° C.

The extraction of the DNA was made using the MoBio PowerSoil™ DNA Isolation kit following the instructions of the manufacturer.

The 16S rRNA gene of the reference strains was amplified using the universal primers 341F (5′-CCTACGGGAGGCAGCAG-3′) (ID SEC N° 3) and 788R (5′-GGACTACCAGGGTATCTAA-3′) (ID SEC N° 4) using the conditions described by Navarrete et al., 2010 (Navarrete P., Magne F., Mardones P., Riveros M., Opazo R., Suau A., Pochart P. and Romero J. 2010. Molecular analysis of intestinal microbiota of rainbow trout (Oncorhynchus mykiss) FEMS MICROBIOLOGY ECOLOGY Volume: 71 Issue: 1 Pages: 148-156). The PCR program consists of 30 cycles at 97° C. for 1 min, 55° c FOR 1 MINUTE and 72° C. for 1 min and 30 s.

The amplified were visualized in gels of polyacrylamide prepared as described by Escanilla and Espejo, 1993. (Detection of HIV1 DNA by a simple procedure of polymerase chain reaction, using primer-dimer formation as an internal control of amplification. Res Virol 144:243-246). The results of the amplification are shown in FIG. 2 that consists of a gel where the amplified fragments are clearly seen and the size can be seen thanks to the markers included in the gel.

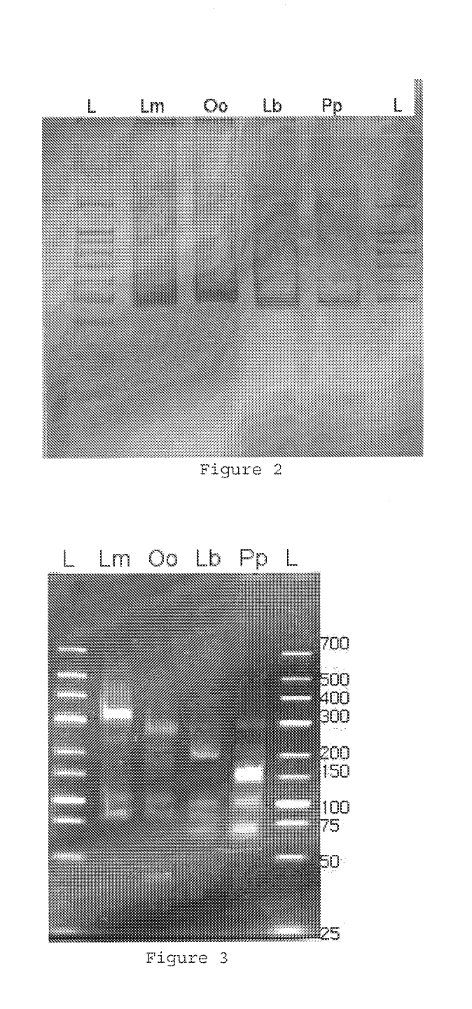

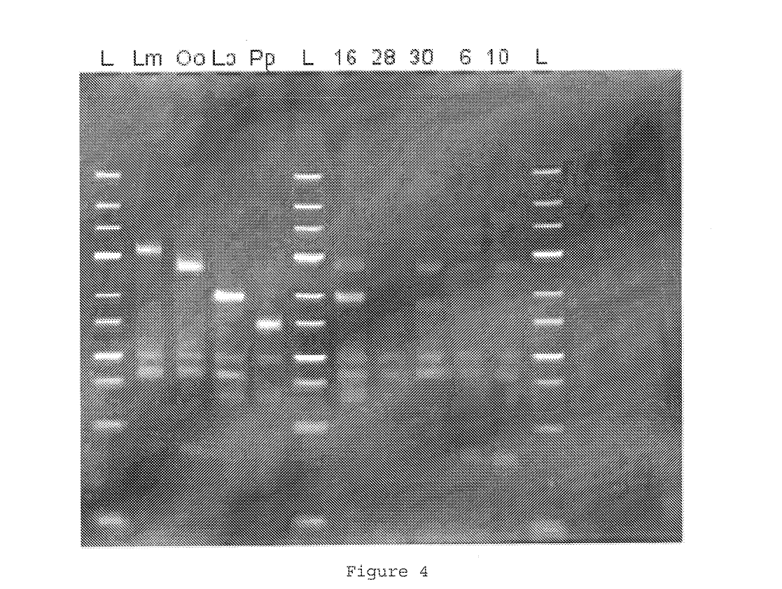

For the restriction analysis enzymes E1 and E2 were used (FIG. 1) selected from the theoretical analysis described above. A double digestion was made at 37° C. during 2 hours and at 65° C. during 10 hours respectively. The profile obtained was visualized in an electrophoresis of a poly acrylamide gel 10% prepared at 80 volts during 90 minutes. The bands were visualized after being dyed with Sybr Green. Table 2 and FIG. 3 show that the expected patterns were obtained.

TABLE 2
Expected digestion

| | Leuconostoc | Oenococcus | Lactobacillus | Pediococcus |
|---|---|---|---|---|
| Strain | mesenteriodes | oeni | brevis | parvulus |
| Digestion | 286 | 255 | 193 | 144 |
| profiles | 79 | 79 | 79 | 127 |
| | 78 | 78 | 78 | 79 |
| | 23 | 31 | 65 | 65 |
| | 0 | 23 | 51 | 51 |
| Addition | 466 | 466 | 466 | 466 |
| of cuts | | | | |

### Example 4

### Confirmation of the Method Analyzing Reference Strains of the Lactic Bacteria Recognized by the Method and Samples of Different Stages in a Wine Fermentation

Samples of different stages of a wine fermentation were taken from the beginning of the alcoholic fermentation until the end of the malolactic fermentation (vinification). The samples were rinsed twice with PVPP (polyvinyl polypyrrolidone) 2% w/v (0.1M EDTA; 0.15M NaCl) to remove the coloring substances. Then the DNA extraction, the amplification, the digestion, the incubation and the visualization in the gel was done such as was described for the reference strains.

FIG. 4 shows the result of the method for the reference strains and samples of the different stages of the fermentation.

The results obtained were patterns clearly distinguishable, obtaining a unique pattern of Oenococcus oeni at the end of the vinification (FIG. 4). These results were contrasted with the technique known as TTGE (Temporal Temperature Gradient Gel Electrophoresis) with the same results obtained (not shown).

1. A method to identify Oenococcus, Leuconostoc, Pediococcus and Lactobacillus bacteria in a fermentation sample comprising the steps of a) obtaining a sample from a fermentation; b) eliminating colorant substances, phenols and other PCR inhibiting substances of the sample; c) lysis of the bacteria present in the sample; d) extracting the DNA from the samples; e) PCR amplification of the DNA extracted using universal primers designed for a target region of the 16S rRNA gene, f) digestion of the amplified using a restriction solution with enzymes selected by theoretical analysis; g) incubation; h) electrophoresis of the fragments obtained.

2. The method of claim 1 wherein the sample of step a) corresponds to an alcoholic fermentation sample of red wine, white wine, rose wine, either regular or sparkling, malolactic fermentation of red wine, white wine, rose wine, either regular or sparkling, fruits fermentation (grapes and/or apples) to produce cider, chicha and pajarete, fermentation for the production of red wine vinegar, white wine vinegar, rice vinegar, apple vinegar, fermentation of wines and ferments for liquors bases such as pisco, rum, whisky, brandy, grappa and/or beer fermentation.

3. The method of claim 2, wherein the sample of step a) corresponds to an alcoholic or malolactic fermentation of red wine, white wine and/or rose wine either regular or sparkling.

4. The method of claim 3, wherein the sample of step a) corresponds to a malolactic fermentation of red wine.

5. The method of claim 1, wherein step b) of eliminating colorant substances, phenols and other PCR inhibiting substances of the sample is done via filtration, centrifugation, treatment with PVPP (polyvinyl polypyrrolidone), activated charcoal, bentonite, casein, silica, gel, agar, isinglass, albumen, gum arabic, vegetable proteins, dioxide silicon, chelating agents, enzymes or combinations thereof.

6. The method of claim 5 wherein step b) is made adding a centrifuged sample of step a) a solution comprising PVPP (polyvinyl polypyrrolidone) at a concentration between 1 and 2% w/v in an adequate buffer.

7. The method of claim 6 wherein the buffer used for the PVPP solution is 0.1M EDTA, 0.15 NaCl.

8. The method of claim 1 wherein step c) lysis of the bacteria present in the sample is done via osmotic shock, mechanical homogenization (french press, blender, glass beads, embolus), sonification, successive freezing and defrosting, treatment with detergents, with alkali, with enzymes or combinations thereof.

9. The method of claim 8 wherein step c) is done with a treatment with enzymes.

10. The method of claim 9 wherein the treatment with enzymes is done incubating the resultant sample of step b) with at least one glucohydrolase in an adequate buffer.

11. The method of claim 10 wherein the glucohydrolase corresponds to lysozyme.

12. The method of claim 9 wherein the treatment with enzymes is done incubating the resultant sample of step b) with at least one protease in an adequate buffer.

13. The method of claim 12 wherein the protease corresponds to a proteinase-K.

14. The method of claim 9 wherein the treatment with enzymes is done incubating the resultant sample of step b) with at least one glucohydrolase in an adequate buffer and then with at least one protease in an adequate buffer.

15. The method of claim 14 wherein the glucohydrolase corresponds to lysozyme and/or the protease corresponds to proteinase-K, independently one from the other.

16. The method of claim 14, wherein step c) is done incubating the resultant sample of step b) during 15 to 60 minutes at 35-37° C. in a solution that comprises a glucohydrolase in an adequate buffer and then incubating the former during 15 to 60 minutes at 35-37° C. in a solution that includes one protease in an adequate buffer.

17. The method of claim 16 wherein the glucohydrolase corresponds to lysozyme and/or the protease corresponds to proteinase-K, independently one from the other

18. The method of claim 16 wherein the incubations is done during 30 minutes.

19. The method of claim 1 wherein d) extracting the DNA from the samples is done with an extraction kit with the phenol chloroform method, with extraction methods and physical, chemical and/or enzymatic lysis using affinity resins with thermal shock with CTAB (cetyltrimethylammonium bromide) or combinations thereof.

20. The method of claim 19 wherein step d) is done with an extraction kit.

21. The method of claim 1 wherein step e) PCR amplification of the DNA extracted using universal primers designed for a target region of the 16S rRNA gene is done using primers 341F (5′-CCTACGGGAGGCAGCAG-3′) (SEQ ID NO: 3) and 788R (5′-GGACTACCAGGGTATCTAA-3′) (SEQ ID NO: 4).

22. The method of claim 21 wherein step e) is done using the conditions described by Navarrete et al., 2010 (Navarrete P., Magne F., Mardones P., Riveros M., Opazo R., Suau A., Pochart P. and Romero J. 2010. Molecular analysis of intestinal microbiota of rainbow trout (Oncorhynchus mykiss) FEMS MICROBIOLOGY ECOLOGY Volume: 71 Issue: 1 Pages: 148-156).

23. The method of claim 1 wherein step f) digestion of the amplified using a restriction solution with enzymes selected by theoretical analysis is done with a solution that comprises at least one restriction enzyme (E1) that recognizes the sequence GAANNNNTTC (SEQ ID NO: 1) and cleaves between the fifth and sixth nucleotide of the recognition sequence in both strands and at least one restriction enzyme (E2) that recognizes the sequence NNCASTGNN (SEQ ID NO: 2) and cleaves the strand 5′-3′ two nucleotides after the underlined recognition sequence is finished and strand 3′-5′ two nucleotides before the start of the underlined recognition sequence and an adequate buffer where the enzymes are active.

24. The method of claim 1 wherein step g) of incubation is done in a thermocycler.

25. The method of claim 24 wherein the program of the thermocycler comprises 90 to 120 minutes at 35-37° C. and 120 to 600 minutes at 62-65° C.

26. The method of claim 1 wherein step h) of electrophoresis of the fragments obtained is done in gels of polyacrylamide at 8 to 10%.

27. The method of claim 26 wherein the gels are subject to 70 to 100 volts during 60 to 90 minutes.

28. The method of claim 1 wherein the DNA of representative strains of the bacterial genders that recognize the method are subject to steps d), f), g) and h) of the method in order to serve as comparison standards to identify each of the genders that the method recognizes.

29. A kit to identify the Oenococcus, Leuconostoc, Pediococcus and Lactobacillus bacteria in a fermentation sample comprising i) Solution or discoloring media; ii) At least one solution or lysis medium; iii) PCR master mix; iv) restriction solution; v) DNA standards of strands of the bacterial genders that the method recognizes; vi) instructions.

30. The kit of claim 29 wherein the kit can further comprise media to obtain the sample from a fermentation.

31. The kit of claim 29 wherein the sample corresponds to an alcoholic fermentation sample from red wine, white wine, rose wine either regular or sparkling, malolactic fermentation of red wine, white wine, rose wine either regular or sparkling, fermentation of fruit (grapes and/or apples) to produce cider, chicha and pajarete, fermentation to produce red wine vinegar, white wine vinegar, rice vinegar, apple vinegar, fermentation of wines and ferments for bases of liquors such as pisco, rum, whisky, brandy, grappa and/or beer fermentation.

32. The kit of claim 29 wherein the solution or discoloring medium includes at least one of the following reactive or physical media: physical media to filtrate or centrifuge the sample, PVPP, activated charcoal, bentonite, casein, silica, gel, agar, isinglass, albumen, gum arabic, vegetable proteins, silicon dioxide, chelant agents, enzymes or combinations thereof.

33. The kit of claim 32 wherein the solution or discoloring media comprises PVPP (polyvinyl polypyrrolidone) at a concentration between 1 and 2% w/v in an adequate buffer.

34. The kit of claim 33 wherein the buffer used for the PVPP solution is 0.1M EDTA, 0.15M NaCl.

35. The kit of claim 29 wherein the solution or lysis medium of the kit includes at least one lysis medium or solution that comprises at least one of the following media or reactive: hypotonic solution, glass beads, detergents solution, hydrolytic enzymes solution, or combinations thereof.

36. The kit of claim 35 wherein the lysis medium or solution of the kit comprises hydrolytic enzymes in an adequate buffer.

37. The kit of claim 36 wherein the lysis medium or solution of the kit comprises at least one enzyme of the glucohydrolase type in an adequate buffer and/or at least one enzyme of the protease type in an adequate buffer.

38. The kit of claim 37 wherein the glucohydrolase comprises a lysozyme and/or the protease corresponds to proteinase-K, independently one from the other.

39. The kit of claim 38 wherein the kit includes at least two solutions or lysis media, one that comprises one glucohydrolase in an adequate buffer and another one that comprises a protease in an adequate buffer.

40. The kit of claim 39 wherein the glucohydrolase corresponds to lysozyme and/or the protease corresponds to proteinase-K, independently one from the other.

41. The kit of claim 29 wherein the PCT master mix of the kit comprises at least the universal primers 341F (SEQ ID NO: 3) and 788R (SEQ ID NO: 4).

42. The kit of claim 40 wherein the PCR master mix further comprises Mg, nucleotides and Taq polymerase enzyme in an adequate buffer.

43. The kit of claim 29 wherein the restriction solution of the kit includes at least one restriction enzyme (E1) that recognizes the sequence GAANNNNTTC (SEQ ID NO: 1) and cleaves between the fifth and sixth nucleotide of the recognition sequence in both strands and at least one restriction enzyme (E2) that recognizes the sequence NNCASTGNN (SEQ ID NO: 2) and cleaves the strand 5′-3′ two nucleotides after the underlined recognition sequence is finished and strand 3′-5′ two nucleotides before the start of the underlined recognition sequence and an adequate buffer where the enzymes are active.

44. The kit of claim 29 wherein the DNA standards of the kits of strains of the bacterial genders that the method recognizes comprises the DNA of at least one strain of each gender (Oenococcus, Lactobacillus, Leuconostoc and Pediococcus) and these strains correspond to species that are frequent in the enology fermentations.

45. The kit of claim 44 wherein the DNA standards of the kit correspond to strains Oenococcus oeni (JCM 6125), Pediococcus parvulus (NBRC 100673), Lactobacillus brevis (ATCC 14687) and Leuconostoc mesenteriodes (LMG 8159).

46. The kit of claim 29 wherein the instructions of the kit include at least instructions referred to the incubations, mix for amplification and digestion, PCR program digestion conditions and/or electrophoresis.
